(19) United States
(12) Patent Application Publication
(10) Pub. No.: US 2017/0176270 A1
(43) Pub. Date: Jun. 22, 2017

(54) NOVEL FIBER-OPTIC PRESSURE SENSOR CONFIGURATIONS

(71) Applicants: Gabriel P. Kern, Houston, TX (US); Kenneth M. Bueche, Friendswood, TX (US); Timothy Daugherty, Katy, TX (US)

(72) Inventors: Gabriel P. Kern, Houston, TX (US); Kenneth M. Bueche, Friendswood, TX (US); Timothy Daugherty, Katy, TX (US)

(73) Assignee: Millar Instruments, Houston, TX (US)

(21) Appl. No.: 15/382,012

(22) Filed: Dec. 16, 2016

Related U.S. Application Data
(60) Provisional application No. 62/268,877, filed on Dec. 17, 2015.
(60) Provisional application No. 62/371,496, filed on Aug. 5, 2016.

(51) Int. Cl. G01L 1/24; G01K 11/32; A61B 5/0215; G02B 6/44

(52) U.S. Cl. CPC G01L 1/246; G02B 6/4415; G01K 11/3206; A61B 5/0215

(57) ABSTRACT

A miniature pressure sensor has been designed based on an optical fiber with fiber bragg grating (FBG). The fiber can be attached to a portion of tubing by co-extrusion, braiding, gluing, or other equivalent methods, where the portion of tubing is flexible and a first side is exposed to the fluid/gas being measured (Pₘ) and a second side is exposed to a reference pressure (P_{ref}).

### RELATED APPLICATIONS

This application claims priority to U.S. Provisional Patent Application No. 62/268,877, filed on Dec. 17, 2015 and U.S. Provisional Patent Application No. 62/371,496, filed on Aug. 5, 2016, which are specifically incorporated by reference in its entirety herein.

### FIELD

The disclosure relates generally to pressure sensors. The disclosure relates specifically to fiber-optic pressure sensors.

### BACKGROUND

Current devices measure pressure and meet the small size catheter crossing profile requirements, but these are generally based on microelectromechanical systems (MEMS) or Fabry-Perot sensing interferometer optical systems. Both of these technologies have their own set of limitations.

MEMS sensors are known to have drift (zero offset) related problems over time and are affected by electro-magnetic interference (EMI) such as produced by MRI machines and electrocautery equipment. Silicon based microelectromechanical (MEMs) sensor manufacturing processes face various size, complexity, and configuration limitations.

The Fabry-Perot sensors are not affected by EMI but have offset or zero shifts due to changes in the curvature of the optic fiber which effect pressure readings from the sensor since these types of sensors work on the basis of quantity of reflected light.

Catheters based on microelectromechanical systems or Fabry-Perot sensing interferometer optical systems are vulnerable to 1) drift-related problems and electro-magnetic interference and 2) offset or zero shifts that affect pressure readings from the sensors, respectively.

It would therefore be advantageous to have an alternative to MEMs-based and Fabry-Perot sensors.

### SUMMARY

An embodiment of the disclosure is a pressure sensing device comprising an optical fiber attached to a portion of a tubing; wherein the optical fiber comprises fiber bragg grating; wherein the optical fiber is present in the tubing; wherein a first side of the optical fiber in the interior of the tubing is exposed to a medium to be measured; wherein a second side of the optical fiber in the interior of the tubing is exposed to a reference pressure; and wherein the optical fiber is co-extruded with the tubing. In an embodiment, the tubing is a catheter. In an embodiment, an outer diameter of the catheter is between 150 micrometers and 6 millimeters. In an embodiment, an outer diameter of the catheter is between 0.5 French to 18 French. In an embodiment, an outer diameter of the catheter is between 1 French and 3 French. In an embodiment, an outer diameter of the catheter is between 300 and 1000 micrometers. In an embodiment, the optical fiber is supported by supports. In an embodiment, the supports are formed in place. In an embodiment, the supports are not integral to the tubing. In an embodiment, the supports are attached by another component. In an embodiment, the supports are attached by epoxy. In an embodiment, the optical fiber is comprised of plastic. In an embodiment, the optical fiber is comprised of glass. In an embodiment, the tubing is comprised of a polymer. In an embodiment, the tubing comprises multiple lumens. In an embodiment, the tubing comprises a guidewire. In an embodiment, the pressure sensing device is compatible with MRI measurements. In an embodiment, the optical fiber is between 20 and 125 micrometers in diameter. In an embodiment, the flexible portion of tubing comprises multiple fibers. In an embodiment, the optical fiber comprises multiple fiber bragg gratings. In an embodiment, the pressure sensing device further comprises one or more temperature sensors. In an embodiment, the one or more temperature sensors are based on FBG. In an embodiment, the one or more temperature sensors are located on the same optical fiber. In an embodiment, one end of the tubing is attached to a connector. In an embodiment, at least one optical fiber is present. In an embodiment, greater than one optical fiber is present. In an embodiment, there is a catenary line effect. In an embodiment, a structure is added to overcome the catenary line effect.

An embodiment of the disclosure is a method of measuring pressure comprising inserting the pressure sensing device of claim 1 into a portion of a patient and measuring a pressure within the portion of the patient. In an embodiment, the tubing is a catheter.

An embodiment of the disclosure is a method of manufacture comprising co-extruding the optical fiber with the tubing; wherein the fiber is present within the tubing; cutting the optical fiber and the tubing into a section of a pre-determined length; and utilizing the pre-determined length of optical fiber within tubing to form a pressure sensing device. In an embodiment, the tubing is a catheter. In an embodiment, a location of the fiber bragg grating is marked. In an embodiment, the pre-determined length is between 100 and 4000 mm In an embodiment, the pre-determined length of optical fiber is in an IV line, medical tube, or medical catheter. In an embodiment, the pre-determined length is between 70 cm and 400 cm. In an embodiment, the optical fiber and tubing are cut by a laser. In an embodiment, the optical fiber comprises fiber bragg grating. In an embodiment, tower bragg grating can be used.

The foregoing has outlined rather broadly the features of the present disclosure in order that the detailed description that follows may be better understood. Additional features and advantages of the disclosure will be described hereinafter, which form the subject of the claims.

### BRIEF DESCRIPTION OF THE DRAWINGS

In order that the manner in which the above-recited and other enhancements and objects of the disclosure are obtained, a more particular description of the disclosure briefly described above will be rendered by reference to specific embodiments thereof which are illustrated in the appended drawings. Understanding that these drawings depict only typical embodiments of the disclosure and are therefore not to be considered limiting of its scope, the disclosure will be described with additional specificity and detail through the use of the accompanying drawings in which:

FIG. 1 depicts a cross-sectional view of a catheter pressure sensor.

FIG. 2 depicts a cross-sectional view of a catheter pressure sensor with a thick catheter tubing wall.

FIG. 3 depicts a cross-sectional view of a catheter pressure sensor with reinforced catheter tubing.

FIG. 4 depicts a cross-sectional view of a catheter pressure sensor with an exterior sleeve of a rigid biocompatible material.

FIG. 5 depicts a cross-sectional view of a catheter pressure sensor with an interior insert of a rigid material.

FIG. 6 depicts a cross-sectional view of a catheter pressure sensor with isolation of the reference section and measurement section with a cutaway portion.

FIG. 7 depicts a cross-sectional view of a catheter pressure sensor with isolation of the reference section and measurement section with a cap separating the two sections.

FIG. 8 depicts a front cross-sectional view of a catheter pressure sensor in which the optical fiber is attached to the wall by co-extrusion during tubing manufacture.

FIG. 9 depicts a front cross-sectional view of a catheter pressure sensor in which the fiber is attached to the wall by gluing the sensor in place with a notch as an alignment feature.

FIG. 10 depicts a front cross-sectional view of a catheter pressure sensor in which the fiber is attached to the wall by snapping in place into a trough that mechanically holds the fiber in place.

FIG. 11 depicts a front cross-sectional view of a catheter pressure sensor in which the fiber is attached to the wall by assembling the fiber in place between two or more components.

FIG. 12 depicts a front cross-sectional view of a catheter pressure sensor in a neutral axis configuration with a bisected circle.

FIG. 13 depicts a front cross-sectional view of a catheter pressure sensor in a neutral axis configuration with a circle (lumen) within a circle.

FIG. 14 depicts a front cross-sectional view of a catheter pressure sensor in a neutral axis configuration with a bisected circle with multiple lumens.

FIG. 15 depicts a front cross-sectional view of a catheter pressure sensor in a non-neutral axis configuration in an exterior wrapped position.

FIG. 16 depicts a front cross-sectional view of a catheter pressure sensor in a non-neutral axis configuration with a tube within a tube.

FIG. 17 depicts a front cross-sectional view of a catheter pressure sensor in which the sensor is integrated within a catheter.

FIG. 18 depicts displacement of the fiber attached to a wall separating the measurement and reference sides.

FIG. 19 depicts a wall separating the measurement and reference sides and attached to supports.

FIG. 20 depicts a catheter pressure sensor with a cutaway portion.

FIG. 21 depicts a catheter pressure sensor with multiple lumens with optical fibers attached to supports.

FIG. 22 depicts a catheter pressure sensor in which the fiber is layered in after the membrane.

FIG. 23 depicts a catheter pressure sensor in which a guidewire lumen is present in the tubing wall and an optical fiber is present in a lumen.

### DETAILED DESCRIPTION

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings making apparent to those skilled in the art how the several forms of the disclosure may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary 3^{rd} Edition.

As used herein, the term “atraumatic” means and refers to a medical device or procedure causing a minimal tissue injury.

As used herein, the term “neutral axis” means and refers to the axis in the cross-section of the catheter shaft along which there are no longitudinal stresses and/or strains.

As used herein, the term “French” means and refers to the size of a catheter equal to 3 times the diameter in millimeters.

The manufacturing processes for silicon based microelectromechanical (MEMs) have various size, complexity, and configuration limitations. The sensors disclosed herein are alternatives to MEMs. The following are issues to consider when designing a pressure sensor.
Temperature compensation is likely needed to measure pressure accurately.
Strain due to bending/deflecting must be minimized to measure pressure accurately; various methods to reduce strain are placing a sensor on the neutral axis, increasing the stiffness of the exterior tubing by reinforcement of an insert/sleeve, and floating/cantilevered placement of the sensor.
Permeability of the material separating reference pressure and measured pressure may cause drift during equalization if reference pressure is sealed. Further, volume changes to the reference pressure must be minimized if reference pressure is sealed. A method to overcome theses is to utilize a vent to atmospheric pressure as reference pressure.

In an embodiment, concerns regarding sensors include temperature compensation, strain caused by bending, and permeability. A bend in blood vessels can cause pressure on the catheter.

In fiber bragg grating (FBG) fiber optic systems, the shift within the spectra of the reflected light is observed and quantified in order to determine the physical event to which the sensor is subjected. FBG fiber optic systems do not measure the quantity of reflected light to determine a physical event.

An optical fiber is known to have relatively good tensile strength but can be compromised when bent to small radii. Therefore, the catheter design can leverage the tensile strength to contribute to the overall strength of the catheter while protecting the fiber from small bending radii. In an embodiment, the optical fiber is comprised of glass or plastic. In an embodiment, data is transmitted through the optical fiber. Catheter can be constructed by inserting the fiber through a thick-walled polymeric tube to both provide protection for the fiber and increase the diameter to that which is comparable to the size of the sensor housing and of the finished catheter diameter.

A miniature pressure sensor has been designed based on an optical fiber with fiber bragg grating (FBG). The fiber can be attached to a portion of tubing by co-extrusion, braiding, gluing, or other equivalent methods, where the portion/wall is flexible and a first side is exposed to the fluid/gas being measured (Pₘ) and a second side is exposed to a reference pressure (P_{ref}). In an embodiment, the fiber can be glued to the tubing after extrusion. In an embodiment, the fiber is on the interior of the tubing. In an embodiment, the fiber is on the exterior wall of the tubing. The reference pressure can be a vacuum, atmospheric pressure, or other pressure. The pressure differential between the two (measurement (Pₘ) and reference (P_{ref}) sides) creates a strain on the portion of tubing and fiber. The strain on the FBG can be measured by light interrogation (light sent down the fiber is reflected/refracted based on the strain at the FBG and causes a wavelength shift); this correlates with the pressure differential and can be used as a pressure sensor. In an embodiment, the FBG sensor is sensitive to strain of 1 microstrain. In an embodiment, the FBG sensor is sensitive to 0.1 mmHg pressure differential between Pₘ and P_{ref}. Other embodiments use sensors with different ranges such as strain of one billionth of an inch or one-thousandth of an inch.

Pₘ is the measured pressure. P_{ref} is the reference pressure. ΔP correlates to the strain on the wall. FIG. 19. The P_{ref} provides a control for temperature. The ΔP subtracts out any temperature effect as it would be equal on Pₘ and P_{ref}. In an embodiment, P_{ref} is vented to atmosphere to that the sensor self-compensates for elevation change. In an embodiment, P_{ref} can be atmospheric pressure or a vacuum.

In an embodiment, the device will be co-extruded. In an embodiment, the co-extrusion can be performed by medical tubing manufacturers. Materials and dimensions that are capable of being manufactured can be used simulate strain and calculate accuracy and sensitivity. A prototype with FBG fiber glued onto an extruded tube can be used to measure accuracy and sensitivity.

In an embodiment, the integral pressure sensor can be manufactured more affordably than current pressure sensors. In an embodiment, the sensor can be extruded simultaneously with the catheter. In an embodiment, multiple materials can be co-extruded at the same time. In an embodiment, a length of catheter co-extruded with the sensor is formed and cut into sections. In an embodiment, the sections are 100-4000 mm In an embodiment, a laser cuts the catheter into sections. In an embodiment, the co-extruded catheter and sensor are present on a spool before being cut into sections. In an embodiment, the laser is programmed to make the cuts automatically. In an embodiment, the cuts are based upon the location of the FBGs. In an embodiment, the locations of the FBG can be marked on the fiber. In an embodiment, the laser is programmed to locate the FBG with a vision system. In an embodiment, one end of the catheter is not laser cut. In an embodiment, a connector can be mounted on the proximal end of the catheter.

In an embodiment, the sensor can be slid into the catheter tube. In an embodiment, one end of the catheter can be left open. In an embodiment, fluid can be allowed to flow in and out of the catheter.

In an embodiment, the catheter can be made rigid by adding a rigid material to the interior or exterior of the catheter via an insert, sleeve, and/or braid. In an embodiment, epoxy can be used to make the catheter rigid. In an embodiment, nylon can be used to make the catheter rigid. In an embodiment, the main body and the flexible portion containing the fiber of the catheter can be made of the same material. In an embodiment, the body and the flexible portion of the catheter can be made of different materials. In an embodiment, the catheter can be comprised of nylon, polyurethane, polyethylene, PTFE, polyimide, PVC, nitinol, silicone, thermoplastic elastomer (TPE), PEEK, Pebas, and others. In an embodiment, the catheter is comprised of silicone. In an embodiment, the catheter is comprised of polydimethylsiloxane (PDMS). In an embodiment, there is no silicon in the sensor and catheter. In an embodiment, the catheter body is made of nylon. In an embodiment, the nylon is nylon 12. In an embodiment, the flexible portion can be made of an elastomer. In an embodiment, the flexible portion can be made of an elastomer including but not limited to at least one of nitrile, rubber, neoprene, silicone, viton, PVC, TPE/TPU, EVA, and PEBA.. In an embodiment, the components of the catheter and sensor are not metal. In an embodiment, the components of the catheter and sensor are non-ferric and are therefore MRI compatible. In an embodiment, the catheter is sterile.

In an embodiment, multiple lumens are present in the co-extruded catheter and sensor. In an embodiment, there are 2 lumens. In an embodiment, there are 1-6 lumens present in the catheter. In an embodiment, lumens can be present within other lumens.

In an embodiment, openings can be cut into the catheter at specific locations. In an embodiment, the openings are laser cut. In an embodiment, the flexible portion can be fixed to one or more bridges at various locations. In an embodiment, the one or more bridges provide support to the membrane. In an embodiment, an insert can be used to create bridges and provide stiffness. FIG. 19.

In an embodiment, the manufacture will remove the cladding from a fiber optic fiber. In an embodiment, the manufacture can make the FBG and add cladding over the FBG. In an embodiment, the cladding is comprised of glass. In an embodiment, the cladding is comprised of suitable materials other than glass. Suitable materials will be readily understood by one of ordinary skill in the art.

In an embodiment, the flexible portion in which the sensor is located is comprised of a flexible polymer. In an embodiment, the flexible polymer is an elastomer. Suitable materials will be readily understood by one of ordinary skill in the art.

Smaller fibers provide better sensitivity. In an embodiment, the optical fiber is 20-125 mm in diameter. In an embodiment, the diameter of the optical fiber is greater than 125 micrometers. In an embodiment, the diameter of the optical fiber is 125 micrometers. In an embodiment, the diameter of the optical fiber is 80 micrometers. In an embodiment, the diameter of the optical fiber is 40 micrometers. In an embodiment, the diameter of the optical fiber is 60 micrometers. In an embodiment, the diameter of the optical fiber is between 1 and 40 micrometers.

Linearity over the pressure range is generally required for an accurate sensor. Depending on the pressure range, the fiber could be pre-tensioned. In an embodiment, the fiber is pre-tensioned during co-extrusion. In an embodiment, a support material is coextruded with the fiber to create tension on the fiber during bending. In an embodiment, a support material is co-extruded with the fiber to create compression on the fiber during bending.

In an embodiment, the catheter can have an outer diameter (OD) of 350 micrometers. In an embodiment, the OD of the catheter can be between 150 micrometers and 6 millimeters. In an embodiment, the OD of the catheter can be between 300 micrometers and 1 millimeter. In an embodiment, the ID of the catheter can be between 0.000001 micrometers and 100 micrometers. In an embodiment, the catheter can have an inner diameter (ID) of 25 micrometers. In an embodiment, the ID of the catheter can be between 12 micrometers and 3 millimeters. In an embodiment, the ID of the catheter can be between 25 micrometers and 650 micrometers. In an embodiment, the catheter is smaller than one French (333 micrometers). Appropriate ID and OD are design characteristics that will readily understood by one of ordinary skill in the art.

In an embodiment, there is a concern regarding bending of the sensor. In an embodiment, the concern can be addressed by keeping the fiber close to a neutral axis. In an embodiment, the material comprising the catheter is stiff so that the catheter does not bend. In an embodiment, there is a metal sleeve on the catheter. In an embodiment, the sensor or catheter can be floated in another device.

In an embodiment, the catheter is used for a right heart catheterization (Swan-Ganz). In an embodiment, the sensor can be used on all Swan-Ganz catheters. In an embodiment, the sensor can be integrated into any type of catheter. In an embodiment, the sensor is present in a right heart catheter. In an embodiment, the sensor is present in a pulmonary catheter. In an embodiment, the sensor can be placed in a medical device other than a catheter. In an embodiment, the device is a circulatory assist device, intraortic balloon pump, pressure enabled guidewire, needletip device, or others. In an embodiment, the sensor can be integrated into any pressure sensing device.

In an embodiment, the sensor can have an internal support. In an embodiment, the support will be provided by rigid material. In an embodiment, the support will be at both ends of the sensor. In an embodiment, the support material will be thermoplastic. In an embodiment, the support material will be nylon, stainless steel, nitinol, titanium, epoxy, PEEK, LCP, PPS, acrylic, and others. However, suitable materials are a matter of design characteristic that will be readily understood by one of ordinary skill in the art.

In an embodiment, there can be two sensors in line. In an embodiment, there can be more than two sensors in line.

In an embodiment, the ends of the catheter are not sealed. In an embodiment, blood can flow through the catheter. In an embodiment, the ends of the catheter are sealed. In an embodiment, only one end of the catheter is sealed. In an embodiment, the cutaway portion is sealed. In an embodiment, the cutaway portion is not sealed. In an embodiment, a two-part sealant can be used such as silicone or epoxy. Examples of biocompatible sealants include, but are not limited to those qualified under USP class VI or ISO 10993 testing. However, other embodiments may use other sealants not qualified by these standards.

In an embodiment, the catheter can be implanted short-term for less than 24 hours or less than 30 days. Durations of time for short term could be a few minutes to a few days, such as 0.1 minute to 7 days or 1 minute to 5 days or 5 minutes to 3 days or 1 hour to 1 day. In an embodiment, the catheter can be implanted long-term. In an embodiment, the catheter can be implanted greater than 30 days. Durations of time for long term could be a few days to permanently, such as one day to permanent or 7 days to 3 years or 2 weeks to 1 year or 1 month to 6 months.

In an embodiment, the catheter is a vent catheter. In an embodiment, the catheter is a left heart vent catheter.

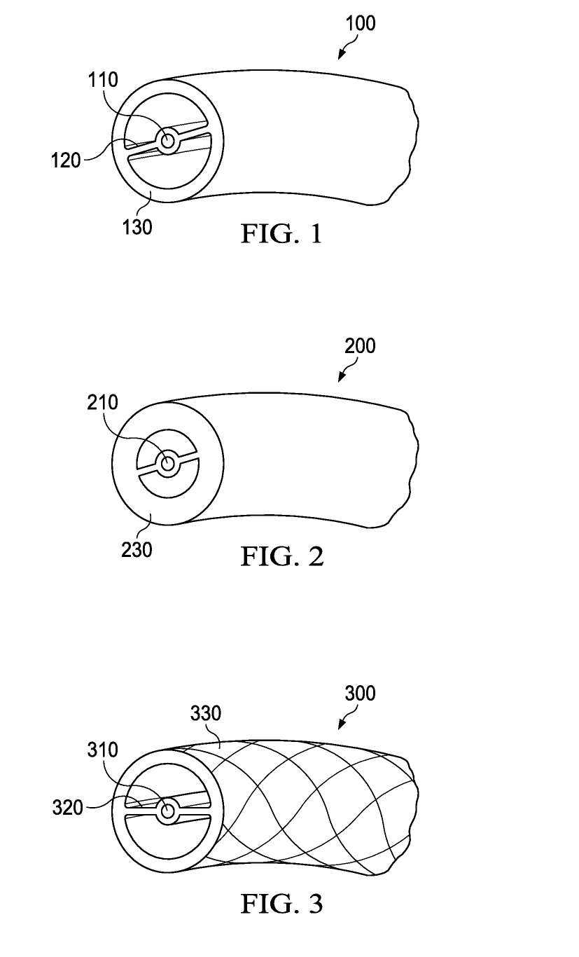

FIG. 1 depicts a cross-sectional view of a catheter pressure sensor (100, 110, 120, 130).

The pressure sensor disclosed compensates for thermal effects and mechanical strain. The pressure sensor can expose one side of the flexible portion to the measurement medium. The pressure sensor can isolate the measurement medium from the reference pressure side of the flexible portion. As an integrated configuration, the pressure sensor can be within another catheter as a miniature pressure sensor that is integral to the catheter while retaining catheter functionality.

### Thermal and Mechanical Strain

All sources of strain on the FBG effect the measurement; sources of strain such as thermal or mechanical strain can interfere with the accuracy of a pressure sensor. In an embodiment, another portion of the fiber with a second FBG can serve as a temperature sensor for temperature compensation to overcome interference. Further, other options are available to resist mechanical strain.

In one configuration, the fiber may be located on the neutral axis, which experiences minimal strain during bending of the catheter. In one embodiment, there can be a rigid portion(s) to resist bending strain; several embodiments are listed below.

### Rigid Portions to Resist Bending/Deflect Strain.

1. Thick catheter tubing wall. FIG. 2 (200, 210, 230).

2. Braided or reinforced catheter tubing, either along the entire length or portion of the length. FIG. 3. (300, 310, 320, 330)

3. Exterior sleeve of metal/plastic/ceramic or other rigid biocompatible material. FIG. 4. (410, 420, 430, 440)

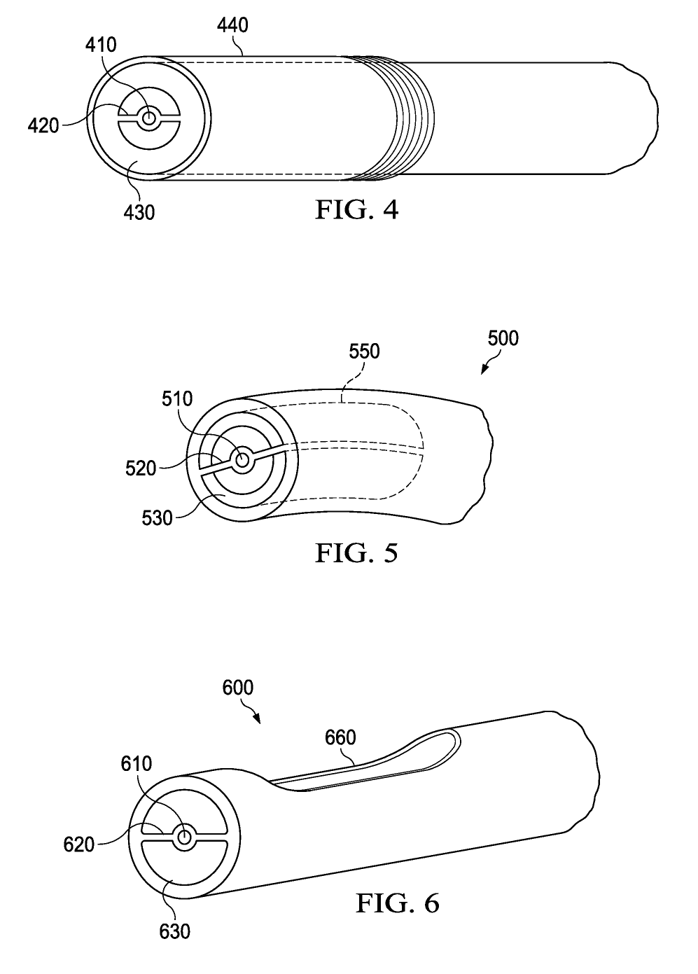

4. Interior insert of metal/plastic/ceramic or other rigid material. In this embodiment, biocompatibility is not necessary. FIG. 5. (500, 510, 520), 530)

5. Formed in place epoxy or other similar material applied to the exterior or interior.

In another configuration, the sensor can be “floating” or cantilever within a housing where the housing would experience the bending/deflecting strain.

### Access to Measurement Medium and Isolation of Reference Pressure and Measurement Medium

1. Cutaway portion of catheter. FIG. 6. (600, 610, 620, 630, 660)

2. Lumen or tubing allowing Gas/Fluid access to one side.

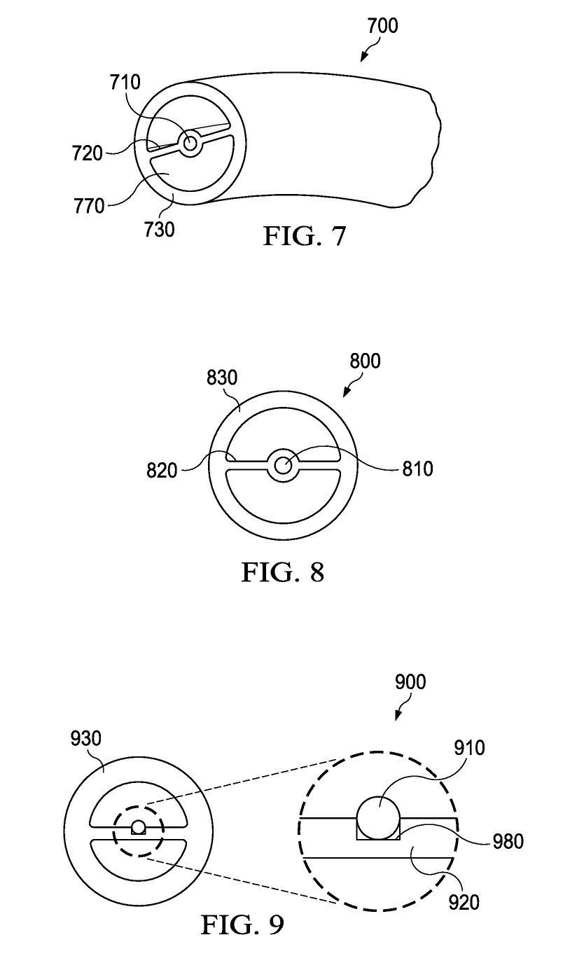

3. Plug or cap separating the two sections. FIG. 7. (700, 710, 720, 730, 770) Attachment (Fiber to Flexible Portion) Methods

1. Co-Extruded together during tubing manufacture. FIG. 8. (800, 810, 820, 830)

2. Glued in place (designed with alignment features such as a notch, hole, lip, etc.). FIG. 9. (900, 910, 920, 930, 980)

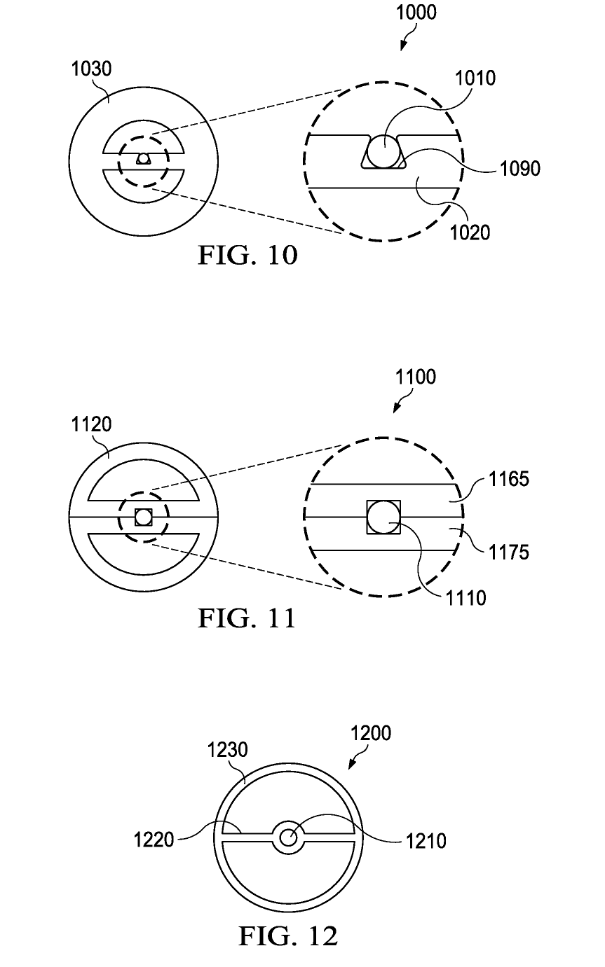

3. Snap in place (designed with lip or trough features that mechanically hold fiber in place). FIG. 10. (1000, 1010, 1020, 1030, 1090)

4. Assembled in place (designed to fit between two or more components). FIG. 11. (1100, 1110, 1120, 1165, 1175)

### Cross Section Profiles

1. Neutral Axis configurations

a. Bisected Oval/Circle. FIG. 12. (1200, 1210, 1220, 1230)

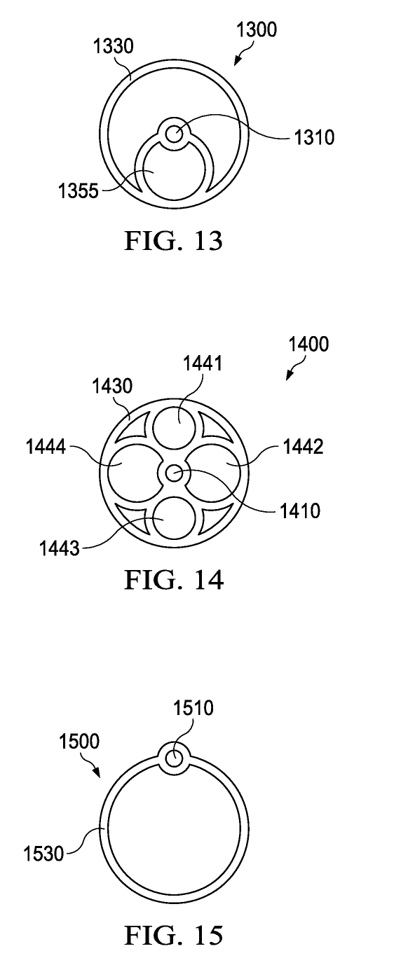

b. Oval/Circle within a Circle. FIG. 13. (1300, 1310, 1330, 1355)

c. Bisected Oval/Circle with 1, 2, or multiple lumens. FIG. 14. (1400, 1410, 1430, 1441, 1442, 1443, 1444)

2. Non-Neutral Axis configurations

a. Exterior wrapped. FIG. 15. (1500, 1510, 1530)

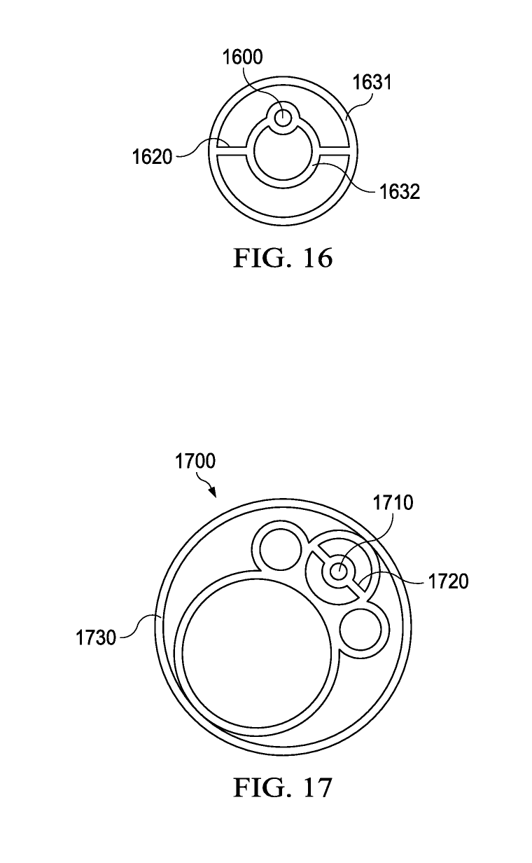

b. Tube within a tube. FIG. 16. (1600, 1620, 1631, 1632)

3. Integrated within a catheter

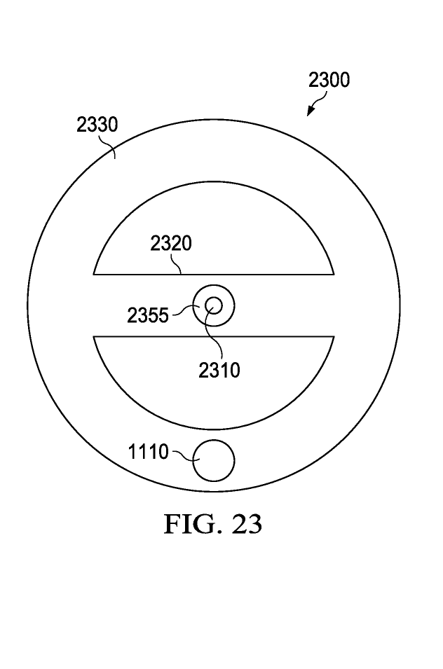

In an embodiment, there can be a guidewire lumen. In an embodiment, the guidewire is present in the measurement medium lumen. In an embodiment, the guidewire is present in a separate lumen. FIG. 17. (1700, 1710, 1720, 1730) In an embodiment, there is a guide wire lumen present in the wall of the catheter. FIG. 23. (2300, 2310, 2320, 2330, 2340, 2355)

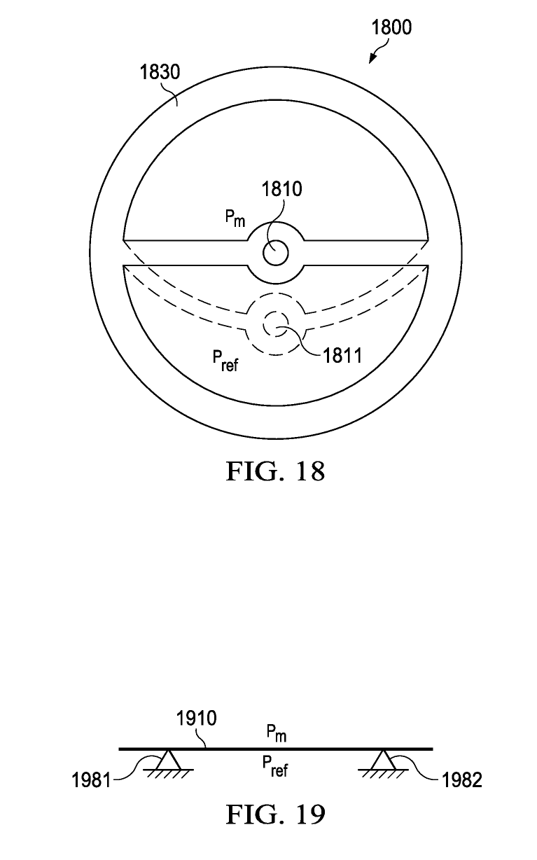

FIG. 18 (1800, 1810, 1811, 1830) depicts displacement of the fiber attached to a flexible portion separating the measurement and reference sides.

FIG. 19 (1910, 1981, 1982) depicts a flexible portion separating the measurement and reference sides and attached to supports.

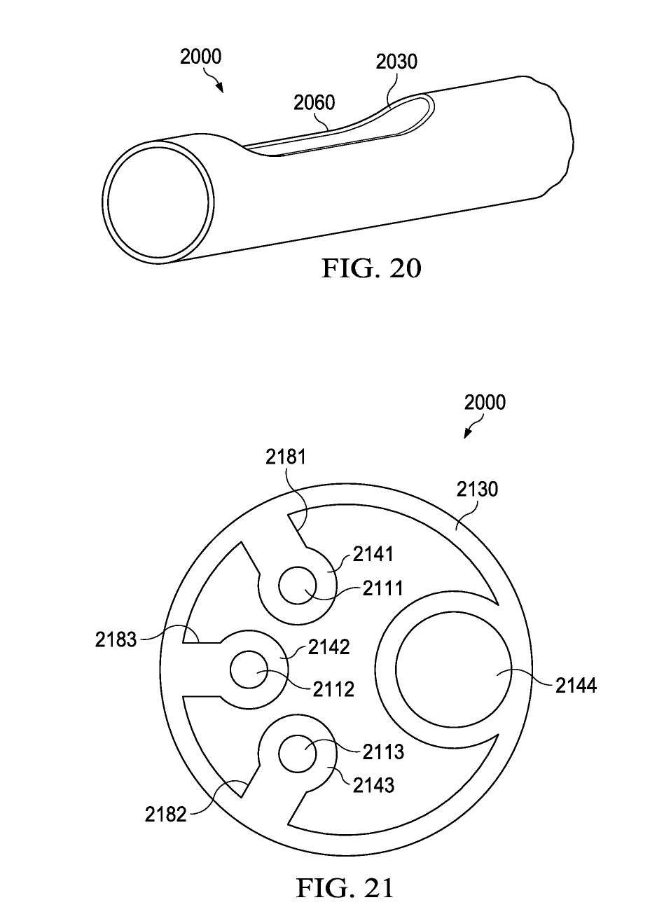

FIG. 20 (2000, 2030, 2060) depicts a catheter pressure sensor with a cutaway portion.

FIG. 21 (2100, 2111, 2112, 2113, 2130, 2141, 2142, 2143, 2144, 2181, 2182, 2183) depicts a catheter pressure sensor with multiple lumens with optical fibers attached to supports.

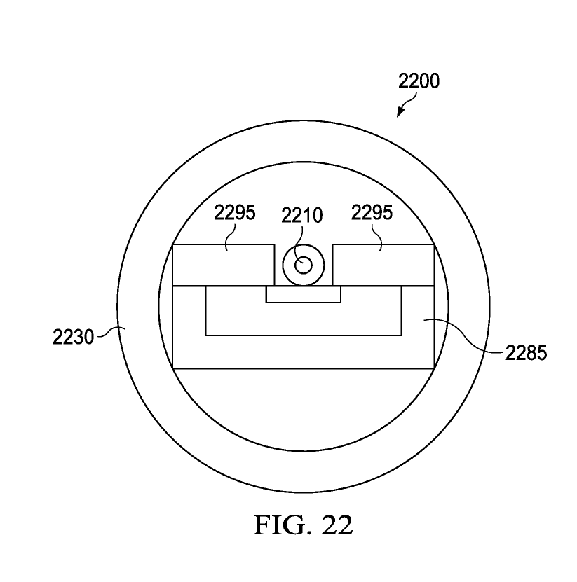

A catheter pressure sensor in which the fiber is layered in after the membrane. FIG. 22. (2200, 2210, 2230, 2285, 2295)

FIG. 23 depicts a catheter pressure sensor in which a guidewire lumen is present in the tubing wall and an optical fiber is present in a lumen.

### Advantages

1. Miniaturization of fiber bragg grating (FBG)-based pressure sensor.

2. Provides a simplified design (reduced components, complexity, assembly operations, handling, etc.) for extrusions of catheters, tubing, lumens, etc.

3. The miniature and simplified pressure sensor can be incorporated into many catheters of various function, without compromising function; faster and easier to measure pressure with a “built in” sensor. This eliminates the need to prepare fluid filled column or to deploy a second pressure measurement device.

4. The neutral Axis configuration reduces or eliminates mechanical strain.

5. Rigid/Semi-Rigid reinforcement reduces or eliminates mechanical strain and may increase pressure induced strain on FBG for increased sensitivity.

In an embodiment, the miniature FBG-based pressure sensor is a non-metallic pressure sensor for MRI safety or other magnetic/electric field compatible applications In an embodiment, the main body of the tubing or housing reduces/eliminates bending strain effects on the FBG portion(s) of the fiber. The pressure sensor can be used within electric/magnetic fields (such as those found in MRIs, electrocautery, cardiac pacing leads, electromagnetic pumps/motors, among others) without the interference typical of MEMs sensors.

Integrated pressure sensor in single or multi-lumen catheters, such as Multi-Lumen Access Catheters, Balloon Wedge Catheters, among others allows quicker/easier diagnostic pressure measurement during procedures while utilizing the basic function of the catheter. As stated above, this removes the need to prepare a fluid filled column or deploy a second device to measure pressure while catheterized.

These configurations can replace catheter pressure sensor technology for medical applications and improve upon applications requiring accurate measurements in the presence of electronic/magnetic fields. The integrated design improves existing catheters by eliminating the step of fluid filled preparation or secondary sensor deployment.

The device provides miniaturization through a simplified design. This design eliminates many of the extra layers, materials, and complexity required to create strain on a fiber and resist mechanical strain. It may be possible to use continuous manufacturing by co-extrusion of the fiber and tubing to significantly reduce cost.

The pressure sensors have a small size, low cost, and high accuracy for medical applications. There is currently not a collinear fiber and tubing configuration to convert pressure differential into strain on fiber and facilitate miniaturization. The miniature size of the pressure sensor can be safely used in small areas of the body including but not limited to the circulatory system and respiratory system. Micro-sized simple sensors could be integrated into most any medical catheterization technologies where pressure measurement may be desired which would simplify procedures. It can allow diagnostic pressure measurements during routine catheterization procedures more frequently. The pressure sensors have stable long-term implantation. The pressure sensors are also easier and less-expensive to manufacture than current sensors.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

1. A pressure sensing device comprising
an optical fiber attached to a flexible portion of a tubing;
wherein the optical fiber comprises fiber bragg grating;
wherein the optical fiber is present in the tubing;
wherein a first side of the optical fiber in the tubing is exposed to a medium to be measured;
wherein a second side of the optical fiber in the tubing is exposed to a reference pressure; and
wherein the optical fiber is attached to the tubing.

2. The pressure sensing device of claim 1 wherein the tubing is a catheter.

3. The pressure sensing device of claim 2 wherein an outer diameter of the catheter is between 150 micrometers and 6 millimeters.

4. The pressure sensing device of claim 3 wherein an outer diameter of the catheter is between 300 and 1000 micrometers.

5. The pressure sensing device of claim 1 wherein the optical fiber is supported by supports.

6. The pressure sensing device of claim 5 wherein the supports are formed in place.

7. The pressure sensing device of claim 1 wherein the optical fiber is comprised of plastic.

8. The pressure sensing device of claim 1 wherein the optical fiber is comprised of glass.

9. The pressure sensing device of claim 1 wherein the tubing is comprised of a polymer.

10. The pressure sensing device of claim 1 wherein the tubing comprises multiple lumens.

11. The pressure sensing device of claim 1 wherein the tubing comprises a guidewire.

12. The pressure sensing device of claim 1 wherein the pressure sensing device is MRI compatible.

13. The pressure sensing device of claim 1 wherein the optical fiber is between 20 and 125 micrometers in diameter.

14. The pressure sensing device of claim 1 wherein the flexible portion of tubing comprises multiple fibers.

15. The pressure sensing device of claim 1 wherein the optical fiber comprises multiple fiber bragg gratings.

16. The pressure sensing device of claim 1 further comprising one or more temperature sensors.

17. The pressure sensing device of claim 16 wherein the one or more temperature sensors are based on FBG.

18. The pressure sensing device of claim 17 wherein the one or more temperature sensors are located on the same optical fiber.

19. The pressure sensing device of claim 1 wherein one end of the tubing is attached to a connector.

20. The pressure sensing device of claim 1 wherein at least one optical fiber is present.

21. The pressure sensing device of claim 1 wherein greater than one optical fiber is present.

22. A method of measuring pressure comprising
inserting the pressure sensing device of claim 1 into a portion of a patient; and
measuring a pressure within the portion of the patient.

23. The method of measuring pressure of claim 22 wherein the tubing is a catheter.

24. A method of manufacture comprising
co-extruding the optical fiber with the tubing;
wherein the optical fiber comprises fiber bragg grating;
wherein the optical fiber is present within the tubing;
cutting the optical fiber and the tubing into a section of a pre-determined length; and
utilizing the cut pre-determined length of optical fiber within tubing to form a pressure sensing device.

25. The method of manufacture of claim 24 wherein the tubing is a catheter.

26. The method of manufacture of claim 24 wherein a location of the fiber bragg grating is marked.

27. The method of manufacture of claim 24 wherein the pre-determined length is between 1000 and 1200 mm.

28. The method of manufacture of claim 24 wherein the optical fiber and tubing are cut by a laser.
